(19) United States
(12) Patent Application Publication
(10) Pub. No.: US 2014/0073698 A1
(43) Pub. Date: Mar. 13, 2014

(54) PHARMACEUTICAL COMPOSITION COMPRISING KETOPROFEN

(71) Applicant: Aventis Pharma S.A., Antony (FR)

(72) Inventors: Sudhakara Rao BADABHAGNI, Guntur (IN); Nilesh JAISWAL, Bhopal (IN); Praveen KHULLAR, Dona Paula (IN); Kum PRASAD, Chanda Nagar (IN)

(73) Assignee: Aventis Pharma S.A., Antony (FR)

(21) Appl. No.: 14/082,408

(22) Filed: Nov. 18, 2013

Related U.S. Application Data
(63) Continuation of application No. PCT/EP2012/059161, filed on May 16, 2012.
(60) Provisional application No. 61/499,842, filed on Jun. 22, 2011.

(30) Foreign Application Priority Data
May 20, 2011 (IN) 1730CHE2011
Jul. 13, 2011 (EP) 11305920.8

(51) Int. Cl. A61K 31/192

(52) U.S. Cl. CPC A61K 31/192
USPC 514/570

(57) ABSTRACT

The present invention relates to a pharmaceutical formulation of ketoprofen sodium salts in a hydrophilic solvent system suitable as a liquid fill composition.

In another aspect, the invention also relates to a process for the preparation of a pharmaceutical formulation and the use of the formulation for treating inflammatory pains.

### FIELD OF THE INVENTION

The present invention relates to a stable pharmaceutical liquid composition of ketoprofen sodium salts for oral administration.

In particular, the invention pertains to an improved composition comprising ketoprofen sodium salts and pharmaceutically acceptable excipients, optionally encapsulated in a capsule.

The present invention furthermore also relates to a process for the preparation of such pharmaceutical composition and the used of said composition for preparing a medicament to treat inflammatory pains.

### BACKGROUND OF THE INVENTION

Ketoprofen [2-(3-benzoylphenyl)propionic acid] is a non-steroidal anti-inflammatory drug (NSAID) that has long been recognized since 1972 being useful in the treatment of fever, pain and inflammation attributed to its analgesic, anti-pyretic and anti-inflammatory properties. Its molecular formula is C₁₆H₁₄O₃ with molecular weight of 254.29. The mechanism of action of ketoprofen is mainly associated to the inhibition of the body's ability to synthesis prostaglandins. Ketoprofen is usually formulated and administered as a racemic mixture of R and S enantiomers, which are equivalent on a per weight basis.

Ketoprofen has certain advantages over aspirin, acetaminophen, and ibuprofen in being a far more effective analgesic and playing a vital role has gained wider acceptance. Ketoprofen is generally taken orally in the form of normal tablets or tablets covered with coatings resistant to gastric juices, or rectally, or by injection, or topically. Ketoprofen is commercially available in market in tablet form under the trade name Orudis KT®. Conventional dosage forms of this drug, administered orally, are rapidly and almost completely absorbed from the gastro-intestinal tract, the peak plasma concentrations occurring within 1 to 3 hours.

For drugs falling into category of NSAID's like ketoprofen and its pharmaceutically acceptable salts it is important that the drug remains in either in solution stage or in colloidal particle size range once reaches the stomach. This helps in absorption of the drug faster and provides earlier onset of action, which is not the case in conventional tablet dosage forms because once the tablet is reached into the stomach it disintegrates into granules and the granules are further disintegrated and the drug exposes to the acid conditions of the stomach. Due to the following sequence of activities there is lag time created which retards the earlier absorption of the drug. The delay in the absorption or higher lag time of absorption is not acceptable for ailments wherein faster onset of analgesic action is expected to benefit the patient.

FR2660555 describes soft gelatin capsules for oral administration comprising ketoprofen sodium salts into dispersion in a fat-based carrier. A disadvantage of the use of theses fat-based carriers is that the composition is not a clear and true solution and thus a dissolution phase of the drug in the digestive apparatus is needed for increasing the onset of action of ketoprofen.

U.S. Pat. No. 5,624,682 discloses a ketoprofen solution in soft capsules which comprises ketoprofen acid dissolved in polyethylene glycol containing a neutralizing organic amine in amount to bring the pH of the solution to between 5 and 7. However, this pharmaceutical composition is not stable over the time and lead to the formation of a high level of impurities from ketoprofen.

It is thus an object of the present invention to develop a pharmaceutical composition for ketoprofen sodium salts that provides a stable composition with an earlier or comparable onset of action compared to the existing prior arts.

Applicants have found that this object can be achieved by providing an improved composition comprising ketoprofen sodium salts and pharmaceutically acceptable liquid hydrophilic carrier medium.

While designing such liquid carrier medium, the carrier medium with the active agent has to be rationally designed, accurately prepared and even slight variations in the composition cannot be tolerated without irreversibly upsetting the system and destroying its beneficial properties. For example, the solubilizing properties of the composition changes and the active substance precipitate out in the event of slight variation in the carrier medium. The applicants have found that encapsulation of such liquid formulations in soft gelatin capsules potentially offers convenient way of administering such pharmacologically active substances without variation in the carrier medium.

Furthermore, oral administration of ketoprofen can cause serious adverse effects most notably gastrointestinal disturbances such as dyspepsia, gastroduodenal bleeding, gastric ulcerations, and gastritis, particularly after extended use. This is because the drug in the tablet dosage form remains in finely divided state in the stomach which activates the AT-pase proton pump due to which gastric disturbances are triggered. Therefore, designing the formulation using specific carrier systems wherein the drug remains dissolved in a solution state helps counter act the gastric acidity. Ketoprofen syrup form available in commercial market is not preferable in terms of patient compliance since ketoprofen has a bitter taste.

The present inventors have recognized this need and have endeavored to circumvent these problems by developing a rationally designed formulation for rapid release of ketoprofen sodium salts. Ketoprofen in a capsule form, apart from the above mentioned advantages, also exhibits more patient's compliance since that taste of the residual remnants of the medicament is masked upon swallowing. Therefore gelatinous capsule forms of ketoprofen have far wider commercial acceptance than their former delivery methods.

### SUMMARY OF THE INVENTION

Thus, the present invention relates to an orally administrable stable pharmaceutical composition, comprising pharmaceutically effective amount of ketoprofen sodium salts, one or more hydrophilic carriers, optionally one or more buffers and/or acidifiers or alkalizers to produce a palatable and stable formulation with rapid therapeutic action.

It is yet another object of the present invention to provide a pharmaceutical formulation, for instance, a soft capsule, a hard capsule, two-piece capsule or tablet comprising the formulation of the instant invention.

Another aspect of the invention is to provide a method for preparing the oral pharmaceutical composition of the invention, comprising dissolving a drug in an appropriate amount of a liquid hydrophilic carrier and bringing the pH to an acceptable range whereby the storage stability and the shelf-life of the formulation is enhanced.

The present invention is also directed to the use of a composition according to the invention for the preparation of a medicament for treating inflammatory pains.

### BRIEF DESCRIPTION OF THE DRAWING

FIG. 1 is a graph showing in vivo bioavailability of a soft gel capsule in accordance with the present invention compared to a tablet formulation.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedence over any dictionary or extrinsic definition. Unless otherwise required by context, singular terms shall include the plural and plural terms shall include the singular.

According to the invention, a stable formulation means a formulation which, in particular, exhibits high resistance against recrystallization or decomposition of ketoprofen sodium salts. Thus, upon storage during 1 or 2 months at 40 deg. C. and 75% humidity, the pharmaceutical composition according to the present invention does not exhibit any sign of crystallinity and remains a clear solution of ketoprofen base. Furthermore, it means that the pharmaceutical composition according to the present invention, upon storage during 1 or 2 months at 40° C. and 75% humidity, contains at least 99% (w/w) of the initial ketoprofen sodium salts and does not exhibit any sign of high level of decomposition, i.e. the level of total impurities is less than 1% by weight based on the ketoprofen sodium salts (as evidenced by HPLC analysis).

The present invention relates an orally administrable stable pharmaceutical composition, comprising pharmaceutically effective amount of ketoprofen sodium salts by compositely establishing optimal conditions for enhancing the storage stability and the shelf-life of the drug in the dosage form, such as the co-relation between the drug and the accompanied components, selection of optimal mixing ratio of the respective components and selection of specific carrier medium, water content and pH regulating agents.

Accordingly, the first aspect of the invention relates to a pharmaceutical liquid composition comprising ketoprofen sodium salts.

In the composition of the present invention, the ketoprofen sodium salts is present in amounts ranging from 1% to 15% by weight of the composition. More preferably, the ketoprofen sodium salts is present in amounts ranging from 6% to 15% by weight of the composition.

The carrier used in the present invention is a liquid hydrophilic carrier. The term “hydrophilic carrier”, refers to one or more of those pharmaceutical excipients which when admixed with ketoprofen at pH 7.5 to 10 increase the aqueous solubility of the ketoprofen into the carrier and has an hydrophilic lipophilic balance (HLB) value of from 10 to 18, preferably from 11 to 16. According to the present invention, the hydrophilic carrier includes, but is not limited to polyethylene glycol (such as PEG200, PEG300, PEG400, PEG600, PEG1000, PEG2000, PEG3000, PEG4000, PEG6000, or PEG8000), glycerin, propylene glycol, PEG 40 hydrogenated castor oil, polysorbate, glyceryl cocoate, PEG 6 caprylic/caprylic glycerides, poloxamer, labrafil, caprylocaproyl macrogol-8 glyceride (Labrasol™), polyethoxylated castor oil (Cremophor™ EL), glycerol-polyethylene glycol oxystearate or PEG-40 Hydrogenated castor oil (Cremophor™ RH), ethoxylated fatty alcohols (Brij) and Sorbitan Esters (Spans). Any of the above-mentioned hydrophilic carriers can be used alone or in combination with one or more hydrophilic carriers. In a preferred embodiment of the invention, the liquid hydrophilic carrier medium is polyethylene glycol 600. In another preferred embodiment of the invention, the liquid hydrophilic carrier medium is a combination of polyethylene glycol 600 and caprylocaproyl macrogol-8 glycerides.

In the composition of the present invention, the liquid hydrophilic carrier is present in amounts ranging from 80% to 95% by weight of the composition and more preferably in an amount ranging from 85% to 95% by weight.

In a further aspect, the present invention relates to an oral administrable formulation comprising ketoprofen as the active agent and a carrier wherein the weight ratio of ketoprofen sodium salts to the liquid hydrophilic carrier is preferably from 1:6 to 1:15. In a most preferred embodiment of the invention, this weight ratio is from 1:12 to 1:15.

Another criterion within the present invention is the pH of the ketoprofen sodium salts in a suitable pharmaceutical vehicle, in order to guarantee an appropriate storage stability and shelf-life of the pharmaceutical formulation and to improve its stability and shelf life. The pH of the composition is ranges from 7.5 to 10, and more preferably from 9.2 to 10.0. In the most preferred embodiment, the pH of the composition is of 9.5.

According to the present invention, if it is required, these pH values can be achieved by means of addition of expedient acidifying and basifying agents or in combination with suitable buffers.

In the composition of the present invention, it is preferred that the acidifying and basifying agents is present in amounts ranging from 0% to 1.5% by weight of the composition.

The basifying agent used in the present invention may be selected from calcium carbonate, magnesium hydroxide, gum acacia, dicalcium phosphate, potassium hydroxide, sodium acetate, potassium phosphate, sodium carbonate, etc. and/or their combinations. The inventors observed (see examples) that use of neutralizing organic amine such has the group consisting of ethanolamine does not allow to obtain stable formulation over the time. Preferably, this basifying agent is the potassium hydroxide.

The acidifying agent used in the present invention may be selected from acetic acid, lactic acid, ascorbic acid, citric acid, phosphoric acid, oxalic acid, calcium chloride, ammonium hydroxide, etc. and/or their combinations. Preferably, this acidifying agent is the citric acid.

The invention also exhibits various advantages with respect to the dosing and convenient method of administration. The invention constitutes yet another advantage over the prior art compositions in that the claimed rationally designed oral formulation of ketoprofen with measured components of other excipients and the process of manufacturing the orally administrable tablets are easily scalable. Further, attributed to this rationally designed oral formulation and the process of making the same, the scalability factor does not impact the in-vivo drug performance or its in-vivo release profile.

The exact dose of active agent and the particular formulation to be administered depend on a number of factors, e.g., the condition to be treated, the desired duration of the treatment and the rate of release of the active agent. For instance, the amount of the active agent required and the release rate thereof may be determined on the basis of known in vitro or in vivo techniques, determining how long a particular active agent concentration in the blood plasma remains at an acceptable level for a therapeutic effect.

Another aspect of the invention relates to a method for preparing the pharmaceutical preparation according to the invention, comprising the following successive steps: dissolving the ketoprofen sodium salt in the liquid hydrophilic carriers, with stirring, in order to obtain an homogeneous mixture; and then, if it is required, adjust the pH using sufficient quantity of basifying agent.

Another aspect of the present invention relates to use of a composition according to the invention for the treatment of inflammatory pains. Specifically, the composition according to the invention could be used for relieving pain and inflammation comprising for instance the kind of pains liable to treatment include, but are not limited to toothache, headache (cephalalgia), migraine, abdominal and pelvic pains, rheumatic pains, ankylosing spondylitis, acute articular and periarticular disorders (bursitis, capsulitis, synovitis, tendinitis), fibrositis neuralgia, fever pains, influenza and common cold symptoms, sore throats, lumbalgia, muscular pains (myalgia), wryneck (torticollis), articular pains, leg pains, contusions, sprains, tendinitis, tennis elbow, lumbago, arthralgia, post-traumatic pain, sciatica, bursitis, fibrositis, painful musculo-skeletal conditions, dysmenorrhea, distentions, minor phlebitis, painful conditions of the spinal column (spinalgia), minor sport injuries, varicose pains, varicose inflammations, bruising (hematomas), intense pains of an acute, subacute and chronic nature (postoperative, tumours, myocardial infarction, traumatisms, fractures) and acute pains from diagnostic and therapeutic measures.

In a further aspect, the invention provides for soft gelatin capsules which include a capsule shell comprising gelatin, plasticizers and, if desired or required, further auxiliary materials.

In developing the soft gelatin capsule ketoprofen formulation according to the present invention, it must be recognized that the capsule is a system comprised of the ketoprofen formulation and the gelatin shell used to encapsulate the ketoprofen formulation. As such, not only is the filled ketoprofen formulation preferred to produce the desired pharmacological action but the gelatin shell formulation is also preferred as it must be compatible with the ketoprofen formulation. One skilled in the art would be aware of the potential fill-shell interactions which could result in both physical and chemical capsule instability. Accordingly, the gelatin shell formulation utilized to form the capsule for the ketoprofen formulation is also preferred and is significant to the present invention.

In general, gelatin shell capsule formulation for soft gelatin capsules consist of raw gelatin and one or more ingredients which are added to plasticize the gelatin to produce a capsule to suitable hardness as required by design or by preference. Typical plasticizers include glycerin, sorbitol, sorbitans and mixtures with glycerine, sorbitan anhydrides and mannitol may also be utilized. Furthermore, other non-traditional ingredients may also be used to plasticize the gelatin.

As such, the gelatin formulations suitable for use with the ketoprofen formulation of the present invention provide the necessary physical and chemical stability required.

A major problem with gelatin-based formulation is an apparent fall in dissolution upon aging, which is attributed to the cross-linking of gelatin-containing products. The cross-linking causes the formation of a swollen, very thin, tough, rubbery, water-insoluble membrane, also known as pellicle. The pellicle acts as a barrier and restricts the release of the drug. Drugs like ketoprofen sodium salts, have tendency to react with gelatin and induce cross linking owing to which the possibility of fall in dissolution during stability studies is high. The present inventors have found that such cross linking of gelatin is surprisingly overcome by addition of certain weak acids in combination with glycine. In a particular embodiment of the invention, the weak acid employed in the present invention is tartaric acid, citric acid or its combinations thereof. The weak acid may be present in an amount of 0.1 to 1.0% (w/w) basis of the gelatin shall formula.

The preferred gelatin formulation for use in constructing soft gelatin capsules containing the ketoprofen formulation of the present invention includes gelatin in the range of approximately 30% (w/w) to approximately 58% (w/w), preferably, but not limited to 40% to 45% and a plasticizer ranging in amount from approximately 10% to approximately 35%, preferably, but not limited to 15%-25%. Suitable plasticizers for use with the preferred capsule formulation include sorbitol (Special MDF 85 grade Ph. Eur.). When sorbitol alone is utilized as the plasticizer, the amount can range from approximately 15% to approximately 40%.

The capsule formulations can also include other suitable additives such as preservatives and/or coloring agents which are utilized to stabilize the capsule and/or impart a specific characteristic such as color or look to the capsule. Pharmaceutically acceptable preservatives can include, for example, methyl and propyl parabens. Color may be imparted to the gelatin shell using FD&C and/or D&C dyes. Exemplary dyes include but are not limited to Tartrazine yellow, Azura red and the like. Opacifiers, such as titanium dioxide and/or iron oxides, may be employed to color and/or render the capsule opaque.

The invention contemplates use of coating agents which may include both non-functional or enteric coating agents such as cellulose based polymers, film coating agents or other coating agents known to a person of skilled in the art.

The present invention also contemplates the use of other pharmaceutical excipients such as binders, disintegrants, diluents, lubricants, plasticizers, permeation enhancers and solubilizers known to a person skilled in the art. Such other additives include antioxidants, preservatives, chelating agents, complexing agents, viscomodulators, tonicifiers, flavorants, colorants odorants, opacifiers, suspending agents, binders, and mixtures thereof. The amounts of such additives can be readily determined by one skilled in the art, according to the particular properties desired.

The pharmaceutical compositions of the present invention can be prepared by conventional methods well known to those skilled in the art. However, the specific method of preparation will depend upon the ultimate dosage form. The hydrophobic therapeutic agent can be solubilized in one or more carriers. The composition can prepared by simple mixing or stirrer of the components to form a concentrate. The stirring process can be aided by heating, if desired. The hydrophobic therapeutic agent can be present in a first amount solubilized by the carrier, and a second amount in the carrier, as desired. It should be emphasized that the order of addition of the various components is not generally important and may be changed as convenient.

According to the invention the preparation of the pharmaceutical formulation based on ketoprofen sodium salts in soft gelatin capsules in carried out in two stages.

Thereafter, a therapeutically active amount of ketoprofen sodium salts is dissolved in an appropriate amount of a pharmaceutical suitable vehicle, the pH is brought to a acceptable range where the stability is more and the mixture is shaken until a transparent and true solution is achieved. If its should be regarded as beneficial, small amounts of sterile water, propylene glycol, glycerol, preservative or other additives such as are generally used for formulation of pharmaceutical compositions in the form of soft capsules can be added to this solution.

As soon as the basic principles for realization of the pharmaceutical formulations according to the present invention in the form of soft gelatin capsules are understood, the expert of pharmaceutical formulations will have no difficulty at all in adapting the process criteria to the particular needs.

Still another object of the invention is to provide a method of administering a pharmaceutical active ingredient to a host to increase the bioavailability of ketoprofen, which comprises the steps of: a) providing a stable gelatin composition of the invention for oral administration; and b) administering said composition to said host for ingestion, whereby said composition contacts the biological fluids of the body and results in faster onset of action.

The core ingredients of a typical formulation according to the present invention may comprise:
1 to 15% (w/w) of ketoprofen sodium salts;
80 to 95% (w/w) of one or more liquid hydrophilic carriers;
wherein the pH value of the composition is between 7.5 and 10.

The gelatin shell ingredients of a typical formulation according to the present invention may comprise:
35 to 50% (w/w), more preferably 40-44% of gelatin;
15 to 30% (w/w), more preferably 15-25% of sorbitol;
0.1 to 10% (w/w) of coloring agents;
0.1 to 10% (w/w) of tartaric acid;
10 to 50% (w/w) of purified water.

Preferably the drug release rate of the composition of the invention filled into a gelatin capsule, when tested in pH 7.4 phosphate buffer (without enzyme), in 900 ml, at 100 RPM and at 37° C., is more than 85% of the ketoprofen sodium salt in 10 minutes.

In use, the methods and compositions of the present invention contemplates a number of important advantages, including:

Robustness and improved delivery at the targeted site: The compositions of the present invention are unexpectedly robust and the compositions of the present invention unexpectedly provide improved delivery of the therapeutic agent to the absorption site, by minimizing precipitation. This improved delivery is believed to result in faster onset of action of the therapeutic agent.

Versatility: Compositions of the present invention can be carefully tailored and scaled to the polarity and functionality of the therapeutic agents, without compromising the improved solubilization, delivery, and other advantages as described above.

Ease of Preparation: The methods of the present invention provide compositions in which the hydrophobic therapeutic agent is readily solubilized, thereby conserving expensive manufacturing and personnel resources.

In a further aspect of the invention, there is provided a process for the preparation of the pharmaceutical composition. The process comprises incorporating the active ingredient ketoprofen into the carrier. Additionally, if appropriate, suitable pharmaceutical excipients are added to formulate an emulsion of the drug. In a preferred embodiment, a suitable carrier is continuously stirred with the active ingredient. The excipients were subsequently mixed.

Soft gelatin capsules are manufactured using rotary die process utilizing gelatin in a conventional process. Dry gelatin granules are combined with water and suitable plasticizers and the combination is then mixed and heated under vacuum to form a molten gelatin mass. The gelatin mass is held in its molten stage while being formed or cast into films or ribbons on casting wheels or drums. The films or ribbons are fed under the wedge and between rotary encapsulation dies. Within the encapsulation dies, capsules are simultaneously formed in pockets in the dies from the films or ribbons. The composition containing ketoprofen is filled into the soft gelatin capsules using any conventional method. The capsule is then cut and sealed. The seals are formed via a combination of pressure and heat as the capsule is filled and cut.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### EXAMPLES

Although the invention has been described in conjunction with specific embodiments, it is evident that many alternatives and variations will be apparent to those skilled in the art in light of the foregoing description. Accordingly, the invention is intended to embrace all of the alternatives and variations that fall within the spirit and scope of the invention.

In the following Examples, the composition ratio shows weight ratio.

### Example 1

### Formulation of Soft Ketoprofen Capsules

| | | | |
|---|---|---|---|
| Ingredients | Function | mg/caps | % (w/w) |

Core formulation

| | | | |
|---|---|---|---|
| Ketoprofen sodium salts | Active ingredient | 25.0 | 12.50 |
| Polyethylene glycol 400 | Hydrophilic base | 172.5 | 86.25 |
| Potassium hydroxide | Inorganic alkalizer | 2.5 | 1.25 |
| Fill wt. (mg) | | 200 | |

Gelatin shell compositions

| | | | |
|---|---|---|---|
| Gelatin | Polymer for sheet | 19.09 | 42 |
| | formation | | |
| Sorbitol special MDF 85 | Plasticizer | 10.91 | 24 |
| Coloring agent | Color | 0.23 | 0.5 |
| Tartaric acid | Anti-crosslinking agent | 0.34 | 0.75 |
| Coloring agent | Color | 0.23 | 0.5 |
| Purified water | vehicle | 14.66 | 32.25 |

The active agent ketoprofen base is dissolved with PEG glycol 400 in a stirrer under continuous stirring until a clear solution is formed. If required, potassium hydroxide is employed for pH adjustment till pH 7.5-10.0 is obtained. The constituents of the pharmaceutical formulation of the invention have a form appropriate for encapsulation. The formulation was encapsulated in a soft gelatin capsule according to one of the methods known per se to those skilled in the art.

### Example 2

### Formulation of Soft Ketoprofen Capsules

| | | | |
|---|---|---|---|
| Ingredients | Function | mg/caps | % (w/w) |

Core formulation

| | | | |
|---|---|---|---|
| Ketoprofen sodium salts | Active ingredient | 27.16 | 6.8 |
| Polyethylene glycol 600 | Hydrophilic base | 372.84 | 93.21 |
| Fill wt. (mg) | | 200 | |

Gelatin shell compositions

| | | | |
|---|---|---|---|
| Gelatin | Polymer for sheet | 19.09 | 42 |
| | formation | | |
| Sorbitol special MDF 85 | Plasticizer | 10.91 | 24 |
| Coloring agent | Color | 0.23 | 0.5 |
| Tartaric acid | Anti-crosslinking agent | 0.34 | 0.75 |
| Coloring agent | Color | 0.23 | 0.5 |
| Purified water | vehicle | 14.66 | 32.25 |

The active agent ketoprofen base is dissolved with PEG glycol 600 in a stirrer under continuous stirring until a clear solution is formed. The formulation was encapsulated in a soft gelatin capsule according to one of the methods known per se to those skilled in the art.

### Example 3

### Formulation of Soft Ketoprofen Capsules

| | | | |
|---|---|---|---|
| Ingredients | Function | mg/caps | % (w/w) |

Core formulation

| | | | |
|---|---|---|---|
| Ketoprofen sodium salts | Active ingredient | 25.0 | 12.5 |
| Polyethylene glycol 400 | Hydrophilic base | 167.5 | 83.8 |
| Potassium hydroxide | Inorganic alkalizer | 2.5 | 1.25 |
| Purified water | | 5.0 | 2.5 |
| Fill wt. (mg) | | 200 | |

Gelatin shell compositions

| | | | |
|---|---|---|---|
| Gelatin | Polymer for sheet | 19.09 | 42 |
| | formation | | |
| Sorbitol special MDF 85 | Plasticizer | 10.91 | 24 |
| Coloring agent | Color | 0.23 | 0.5 |
| Tartaric acid | Anti-crosslinking agent | 0.34 | 0.75 |
| Coloring agent | Color | 0.23 | 0.5 |
| Purified water | vehicle | 14.66 | 32.25 |

The active agent ketoprofen base is dissolved with PEG glycol 400 in a stirrer under continuous stirring until a clear solution is formed. If required, potassium hydroxide is employed for pH adjustment till pH 7.5-10.0 is obtained. The constituents of the pharmaceutical formulation of the invention have a form appropriate for encapsulation. The formulation was encapsulated in a soft gelatin capsule according to one of the methods known per se to those skilled in the art.

### Example 4

### Comparative Stability Study of the Composition According to the Invention

Different fill compositions (from A to F) were tested. Stress testing were carried out on a single batch of each fill composition including the effect of temperatures (40° C.) for accelerated testing and humidity (75% RH). The testing allows to evaluate the susceptibility of the ketoprofen sodium salt to hydrolysis and specifically to form cyclic ester impurities of ketoprofen.

The stability studies have been performed on the liquid formulation fills filled in glass vials and measurement was made after 1 (1M) and 2 months (2M) after being carried out in such accelerating conditions.
The results obtained are the following:

| | | | Total |
|---|---|---|---|
| Composition A | | | Impurities |
| (comparative example) | mg/fill | Condition | level (% w/w) |
| | | Initial | 0.181 |
| Ketoprofen base | 25.0 | 1M @ 40° C./75% RH | 0.190 |
| PEG 600 | 125.0 | 2M @ 40° C./75% RH | 0.229 |
| Triethanolamine | 5.62 | | |
| pH = 5-7 | | | |

| | | | Total |
|---|---|---|---|
| | mg/fill | Condition | Imp |
| Composition B (comparative | | | |
| example) | | | |
| | | Initial | 0.185 |
| Ketoprofen base | 50 | 1M @ 40° C./75% RH | 0.196 |
| PEG 600 | 250 | 2M @ 40° C./75% RH | 0.231 |
| Triethanolamine | 11.24 | | |
| pH = 5-7 | | | |
| Composition C (comparative | | | |
| example) | | | |
| | | Initial | 0.079 |
| Ketoprofen base | 25.0 | 1M @ 40° C./75% RH | 0.094 |
| PEG 600 | 80.0 | 2M @ 40° C./75% RH | 0.282 |
| PEG 400 | 125.0 | | |
| Triethanolamine | 27.9 | | |
| pH = 5-7 | | | |
| Composition D (comparative | | | |
| example) | | | |
| | | Initial | 0.054 |
| Ketoprofen base | 25.0 | 1M @ 40° C./75% RH | 0.132 |
| PEG 400 | 370.0 | 2M @ 40° C./75% RH | 0.213 |
| Glycerine | 5.0 | | |
| Triethanolamine | 5.6 | | |
| pH = 5-7 | | | |
| Composition E (according to | | | |
| the invention) | | | |
| | | Initial | 0.068 |
| Ketoprofen base | 25.0 | 1M @ 40° C./75% RH | 0.068 |
| PEG 600 | 370.0 | 2M @ 40° C./75% RH | 0.092 |
| Potassium Hydroxide | 5.0 | | |
| pH = 9.5 | | | |
| Composition F (according to | | | |
| the invention) | | | |
| (Test Invention) | | Initial | 0.033 |
| Ketoprofen sodium | 27.16 | 1M @ 40° C./75% RH | 0.031 |
| PEG 600 | 372.84 | 1M @ 40° C./75% RH | 0.033 |
| pH = 9.5 | | | |

In the above tables, the impurities level is expressed by weight based on the ketoprofen sodium salts.

Stability study results show that after 2 months studies conducted at 40° C.±2° C./75% RH±5% RH a significant change occurs in assay from its initial value of the impurities level (from less than 0.1% w/w to a level greater than 0.2% w/w) at the accelerated storage condition for the composition A, B, C and D. Theses degradation product's exceeding the acceptance criterion.

On the other hand, composition E and F according to the invention has a level of total impurities which remains stable, less than 0.1% (w/w) and thus acceptable.

### Example 5

### Comparative In Vitro Dissolution Study and In Vivo Bioavailability of the Composition According to the Invention

The dissolution profile of the composition of the example 3 is compared to the dissolution profile of the Toprec® product 25 mg tablet.

### Methodology:

### A) Dissolution Conditions:

Apparatus: Paddle with sinkers

### Speed: 100 RPM

Dissolution media: 900 ml

### Temperature: 37° C.±0.5

Time points: 10, 15, 30, 45 & 60 minutes or as required time points.

### b) Dissolution Media Composition:

pH 7.4 phosphate buffer (without enzyme)

### c) Test Procedure:

Weigh and drop 1 capsule in each of six dissolution vessels which contains dissolution medium. After specified time point withdraw 10 ml of the aliquot from a zone midway between the surface of the dissolution medium and the top of the rotating blade, not less than 10 mm away from the wall of the vessel. Filter the solution through 0.45 μm nylon 66 filter paper.

### d) Chromatographic Conditions:

Equipment: Liquid chromatograph equipped with UV detector
Column: X′ terra, C18, 150 mm×4.6 mm, 5 μm or equivalent
Column temperature: 35° C.
Flow rate: 1.2 ml/min
Detection UV wavelength: 260 nm

### e) Instrumentation

a. Validated Automated Dissolution System.
b. HPLC System comprising of pump, auto injector, UV detector

### Results:

### In Vitro Dissolution Profile:

The dissolution profile of the composition of the example 2 was tested.

| Time | % (w/w) drug dissolved at pH 7.4 Phosphate |
|---|---|
| points | buffer/900 ml/Paddle with Sinker/100 rpm |
| 10 min | 65 |
| 15 min | 92 |
| 30 min | 93 |
| 45 min | 93 |
| 60 min | 93 |

The above experimental results reveal that 92% (w/w) of the ketoprofen sodium salt dissolved is released after 15 min for a composition according to the invention.

### Pharmacokinetic Simulation:

The pharmacokinetic blood profiles for the pharmaceutical composition of the present invention were calculated using a simulation program.

A mathematical model (Gastroplus™ simulator software available from Simulations Plus, Incorporated) was developed to explore the input rates for ketoprofen dosage forms that would meet certain in vivo release targets. Gastroplus® is a computer program that simulates absorption and pharmacokinetics for orally dosed drugs. The underlying model is the Advanced Compartmental Absorption and Transit (ACAT) model—an extension of work originally done by Gordon Amidon and Lawrence Yu. See L. X. Yu, “An Integrated Model for Determining Causes of Poor Oral Drug Absorption.” Pharm. Res. 16:1883-7 (1999) and B. Agoram, W. S. Woltosz, and M. B. Bolger, “Predicting the impact of physiological and biochemical processes on oral drug bioavailability,” Advanced Drug Delivery Reviews, 50:S41-S67 (2001).

Several different screens within the Gastroplus software were modified to generate the optimum dosing curves.

The Compound screen has the following parameters which can be varied:
Dosage Form: IP solution
Initial Dose (mg): 25 (example)

### Subsequent Doses (mg): 0

Dosing Intervals (hours): 0

### Dose Volumes (mL): 250

pH for ref. Solubility: 2
Solubility (mg/mL @pH=2): 0.118
Mean Precipitation Time (sec): 90
Diffusion Coefficient (cm²×10⁵)=0.432
Drug Particle Density (g/mL): 1.253
Peff—Effective Permeability (cm/s×10⁴): 8.7
Sim Peff×10⁴ (human): 8.7

### Molecular Weight=254.29

Reference Log D=3.16 @pH=1

### Particle Size: R=25.00, D=50.00

pKa: 4.6/SolFactor: 353/FuncGroup: Carboxylic acid
The Physiology screen has the following parameters which can be varied:
Stomach: Peff=0; ASF=0; pH=1.30; Transit Time (hours)=0.25; Volume (ml)=50; Length (cm)=15.0; Radius (cm)=10.00
Duodenum: Peff=0; ASF=1.305; pH=6.00; Transit Time (hours)=0.26; Volume (mL)=48.25; Length (cm)=30.0; Radius (cm)=1.60
Jejunum 1: Peff=0; ASF=1.366; pH=6.20; Transit Time (hours)=0.95; Volume (mL)=175.3; Length (cm)=62.00; Radius (cm)=1.50
Jejunum 2: Peff=0; ASF=1.502; pH=6.40; Transit Time (hours)=0.76; Volume (mL)=139.9; Length (cm)=62.00; Radius (cm)=1.34
Ileum 1: Peff=0; ASF=1.675; pH=6.60; Transit Time (hours)=0.59; Volume (mL)=108.5; Length (cm)=62.00; Radius (cm)=1.18
Ileum 2: Peff=0; ASF=1.906; pH=6.90; Transit Time (hours)=0.43; Volume (mL)=79.48; Length (cm)=62.00; Radius (cm)=1.01
Ileum 3: Peff=0; ASF=2.177; pH=7.40; Transit Time (hours)=0.31; Volume (mL)=56.29; Length (cm)=62.00; Radius (cm)=0.85
Caecum: Peff=0; ASF=0.229; pH=6.40; Transit Time (hours)=4.50; Volume (mL)=52.92; Length (cm)=13.75; Radius (cm)=3.50
Asc Colon: Peff=0; ASF=0.243; pH=6.80; Transit Time (hours)=13.50; Volume (mL)=56.98; Length (cm)=29.02; Radius (cm)=2.50

### C1-C4: 0.18148/1.0944/0.0734/0.31836

ASF Model: Opt logD Model

### Qh (L/min)=1.5

### Percent Fluid in SI: 40. Colon: 10.

The Pharmacokinetics screen has the following parameters which can be varied:
PK model: Compartmental

### Body Weight (kg): 82

Blood/plasma concentration ratio: 1

### Use Exp Plasma Fup (%): 0.75

### Renal Clearance (L/h): 5.04

### Vc (L/kg): 0.15

T ½ (h): 1.69

Gastroplus® was used to simulate the absorption and pharmacokinetics of the reference and test formulations. The in vitro dissolution profiles of ketoprofen hydrochloride formulations were used as input functions to simulate the absorption and pharmacokinetics of the reference (Toprec® 25 mg commercial tablet) and test formulations.

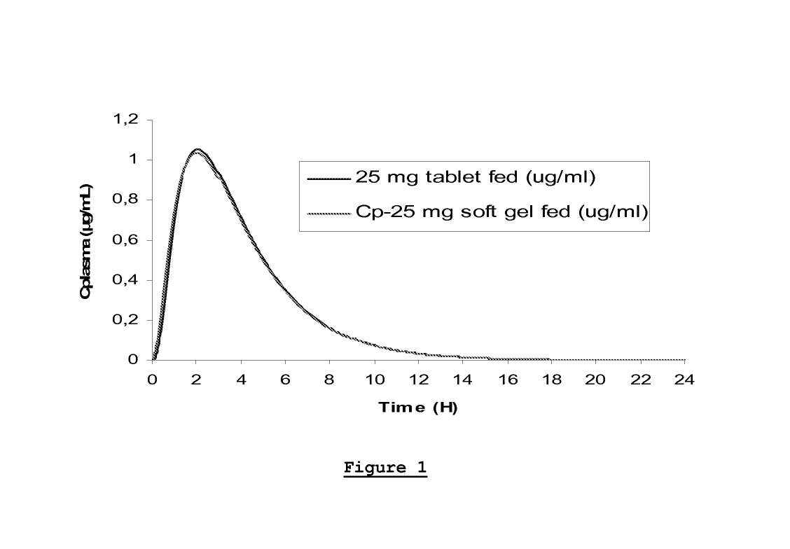

The FIG. 1 shows the results of using the model to simulate in vivo bioavailability in Fed state of the Toprec® 25 mg marketed tablet and of a pharmaceutical composition according to the invention containing 25 mg of ketoprofen sodium salt.

The model was used to evaluate the performance of the dosage forms of the invention. These data show that the dosage forms of the invention will be effective. The AUC ratio (equal to 1 in the Fed state and to 1 in the Fasted state) and the Cmax ratio (equal to 1 in the Fed state and to 1.035 in the Fasted state) were found to be comparable for a soft gel and tablet formulation of ketoprofen sodium salts at 25 mg using the Gastro Plus software.

The foregoing discussion and description are illustrative of some embodiments of the present invention, but are not meant to be limitations on the practice thereof.

What is claimed is:

1. A pharmaceutical composition for oral administration comprising:
1 to 15% (w/w) of ketoprofen sodium salts; and
80 to 95% (w/w) of a liquid hydrophilic carrier medium comprising one or more liquid hydrophilic carriers;
wherein the pH value of the composition is between 7.5 and 10.

2. The pharmaceutical composition according to claim 1, wherein the pH value of the composition is 9.5.

3. The pharmaceutical composition according to claim 1, wherein total amount of the liquid hydrophilic carrier medium ranges from 85 to 95% (w/w).

4. The pharmaceutical composition according to claim 1, wherein the liquid hydrophilic carriers are chosen from the group consisting of polyethylene glycols, glycerin, propylene glycol, PEG 40 hydrogenated castor oil, polysorbate, glyceryl cocoate, PEG 6 caprylic/caprylic glycerides, poloxamer, labrafil, caprylocaproyl macrogol-8 glyceride, polyethoxylated castor oil, glycerol-polyethylene glycol oxystearate or PEG-40 hydrogenated castor oil, ethoxylated fatty alcohols and sorbitan esters.

5. The pharmaceutical composition according to claim 1, wherein the liquid hydrophilic carrier medium is polyethylene glycol 600.

6. The pharmaceutical composition according to claim 1, wherein, if it is required, the pH is adjusted with citric acid.

7. The pharmaceutical composition according to claim 1, wherein, if it is required, the pH is adjusted with potassium hydroxide.

8. The pharmaceutical composition according to claim 1, wherein the weight ratio of ketoprofen sodium salts to the liquid hydrophilic carrier medium is from 1:12 to 1:15.

9. The pharmaceutical composition according to claim 1, in the form of a soft capsule.

10. A method for preparing a pharmaceutical preparation according to claim 1, comprising the following successive steps: dissolving the ketoprofen sodium salts in the liquid hydrophilic carrier medium, with stirring, in order to obtain a homogeneous mixture; and then, if it is required, adjusting the pH using sufficient quantity of an acidifying or a basifying agent.

11. A method for the treatment of an allergic reaction in a patient comprising the administration of a pharmaceutically effective dose of a composition as claimed in claim 1 to the patient.
